(19) United States
(12) Patent Application Publication
(10) Pub. No.: US 2016/0346046 A1
(43) Pub. Date: Dec. 1, 2016

(54) IDENTIFICATION AND CALIBRATION METHOD

(71) Applicant: Brainlab AG, Feldkirchen, DE (US)

(72) Inventors: Tanja Tausch, Munich (DE); Stefan Wimmer, Munich (DE)

(73) Assignee: Brainlab AG

(21) Appl. No.: 15/116,421

(22) PCT Filed: Feb. 5, 2014
(86) PCT No.: PCT/US2014/052221

§ 371 Date: Aug. 3, 2016

(51) Int. Cl. A61B 34/20; A61B 90/00; A61B 5/06

(52) U.S. Cl. CPC A61B 34/20; A61B 5/064; A61B 90/39; A61B 2034/2046; A61B 2090/3954; A61B 2090/3966; A61B 2090/3925; A61B 2090/397; A61B 2090/3937

(57) ABSTRACT

The instant invention describes an anti-biofouling structure for placement onto structures or surfaces that are exposed to aquatic environments. Embedded within the anti-biofouling structure are agents that can diffuse out of the structure and prevent the formation and/or accumulation of plant and animal species build-up that creates biofouling. The instant invention also describes system for preventing biofouling of an object stored in an aquatic environment which includes the anti-biofouling structure and a protective cover element constructed and arranged to fit various structures, such as boat propellers.

The invention relates to the general technical field of calibration methods used to calibrate and identify medical instruments which are tracked by means of a surgical tracking system during image-guided surgery.

In medical procedures such as image-guided surgery it s desirable to know the position of medical instruments relative to the patient to be treated. In order to determine the spatial position (i.e. the spatial location and/or orientation) of the instruments, they are provided with tracking markers which can be spatially tracked by a medical tracking system. However, in order to determine the spatial position of the instrument relative to the tracking markers, calibration procedures have to be performed before the instrument is used in a surgical procedure, wherein the calibration procedure generally consists of moving the instrument to be calibrated in a predetermined manner so that the relative position of the portions of the instrument which are of interest can be determined by a navigation system. The relative position of a pointed instrument tip can for example be determined by pivoting the instrument in several directions while the instrument tip remains at the same position, such that the tracking marker(s) describe/s a sphere around the instrument tip, wherein the instrument tip represents the centre of the sphere, such that the position of the instrument tip relative to the tracking markers can then be calculated.

U.S. 2008/0183387 A1 discloses a marker system and method for determining the diameter and axial location of a cylindrical hole in an instrument which comprises a plurality of tracking markers.

Such calibration procedures are however laborious and time-consuming.

It is the object of the present invention to provide an efficient and reliable method for identifying a medical instrument to be used during computer-assisted surgery.

This object is achieved by the subject-matter of any appended independent claim. Advantages, advantageous features, advantageous embodiments and advantageous aspects of the present invention are disclosed in the following and contained in the subject-matter of the dependent claims. Different advantageous features can be combined in accordance with the invention wherever technically expedient and feasible. Specifically, a feature of one embodiment which has the same or a similar function to another feature of another embodiment can be exchanged with said other feature, and a feature of one embodiment which adds an additional function to another embodiment can in particular be added to said other embodiment.

The invention provides a data processing method for identifying a medical instrument which is configured to be tracked by means of a medical tracking system, wherein the method is designed to be executed by a computer and comprises the following steps:
acquiring relative position and/or movement data which comprise relative position and/or movement information describing a relative position and/or movement between a calibration tool, which comprises at least one supporting surface, and the medical instrument which is in contact with the at least one supporting surface;
acquiring geometry data which comprise geometry information describing at least one geometrical feature for a plurality of candidate instruments;
determining, on the basis of the relative position and/or movement data and the geometry data, candidate data which comprise candidate information describing at least one candidate instrument from the plurality of candidate instruments, the at least one geometrical feature of which would allow the relative position and/or movement between the medical instrument and the calibration tool.

In other words, a calibration tool which is provided comprises at least one supporting surface which can form indents, notches or recesses which allow a corresponding portion of a medical instrument which is to be identified during an identification procedure to be held or guided. The medical instrument is brought into contact with the calibration tool, for example by inserting a portion of the instrument into a notch or recess, and the instrument is held and/or moved relative to the calibration tool, wherein the physical contact between the instrument and the calibration tool is maintained with the aid of the at least one supporting surface. During this procedure, the position of the instrument relative to the calibration tool is determined, for example by determining the position of tracking markers which are attached to the instrument and calibration tool, respectively. However, for the purpose of simply identifying the instrument, it would be sufficient to provide one of the instrument and the calibration tool with tracking markers, while the other—in particular, the oration tool—is held in a known and spatially invariant position.

It will be apparent that holding and/or moving instruments exhibiting different geometries with respect to the same calibration tool will result in different positions and/or movements of the instrument relative to the calibration tool.

In addition to the method step described above, it is possible to provide a database comprising information about the geometry of a plurality of instruments which can be used in connection with a surgical procedure. For each of these instruments, the database comprises a dataset containing information on at least one geometrical feature, for example the shape of the longitudinal axis of an elongated instrument body and/or the position of the instrument's tip relative to the instrument tracking markers.

In accordance with the method of the invention, the data which are obtained while the instrument and calibration tool are held and/or moved relative to each other, and from which conclusions can be drawn regarding the geometry of the current instrument, are compared with the data which are provided by the database and describe geometrical features of a plurality of instruments known to the system.

This comparison allows any known instrument, the geometry of which would not allow the determined relative position and/or movement between the instrument and the calibration tool, to be excluded. The set of candidate instruments is therefore reduced to a set of candidate instruments exhibiting a geometry which would allow the determined relative position and/or movement between the instrument and the calibration tool, by excluding those which exhibit any relative position or relative movement which is impossible to perform using at least one instrument contained in the initial set of candidate instruments. By performing a sufficient number of such relative positions and/or movements, it is possible to reduce the set of candidate instruments down to one candidate instrument dataset corresponding to the actual instrument which has been held and/or moved relative to the calibration tool. It is the possible to perform computer-assisted surgery, by providing the tracking and navigation system with the data contained in the dataset for the one remaining candidate instrument. In other words, the method of the invention allows a particular instrument to be identified from among a plurality of precalibrated instruments.

It will be apparent from the above that the method of the invention for identifying a particular instrument can be performed more easily and quickly than any “complete” calibration method, since the instrument has to be held and/or moved relative to the calibration tool merely in order to exclude candidate instruments until only one remains, whereas a complete calibration requires numerous relative positions and/or movements to be performed in order to identify enough of the geometrical properties of the instruments which have to be known during computer-assisted surgery.

In accordance with one preferred embodiment of the present invention, the calibration tool is configured to be tracked by means of a medical tracking system, wherein the at least one supporting surface is in particular configured to restrict the relative position and/or relative movement between the calibration tool and the medical instrument in contact with the supporting surface.

Although the calibration tool is preferably provided with tracking markers which are configured to be tracked by a medical tracking system, this is—as already mentioned above—not necessary if the spatial position of the calibration tool along with the at least one supporting surface is known when the instrument to be identified is brought into contact with the calibration tool and held and/or moved relative to the calibration tool. Preferably, at least one supporting surface restricts the relative position and/or relative movement between the calibration tool and the instrument. At least one cylindrical recess can for example be provided which restricts any rotational movement of a rotationally symmetrical and elongated instrument, which has been inserted into the recess, to that about the longitudinal axis of the recess and restricts any translational movement to that necessary for inserting/withdrawing the instrument into/from the recess. Another example of such supporting surfaces is a notch which is formed by two angled supporting surfaces which restrict any translational movement of an instrument, which has been inserted into the notch and is in contact with the sides or edges of both supporting surfaces, to the translational movement in which the instrument is drawn through the notch.

In general terms, is possible to use an relative position and/or relative movement of the instrument with respect to the calibration tool to determine geometrical properties of the instrument, on the basis of which it possible to exclude at least one candidate instrument dataset other than the dataset describing the instrument which has actually been brought into contact with the calibration tool and held in a position and/or moved relative to the calibration tool, wherein physical contact between the instrument and the at least one supporting surface of the calibration tool is maintained.

It is also conceivable for the step of acquiring relative position and/or movement data to be performed several times, in particular at different relative positions and/or by performing different relative movements between the calibration tool and the instrument, in particular if the database contains datasets of instruments exhibiting a broadly similar geometry, since at least one geometrical feature has to be determined which allows such similar instruments to be unambiguously distinguished.

It is also preferable for the step(s) of acquiring relative position and/or movement data to be performed until only one candidate instrument from the plurality of candidate instruments remains, the at least one geometrical feature of which would allow all of the relative position(s) and/or movement(s) performed up until that point, since it is only then possible to be absolutely sure that the instrument currently in contact with the calibration tool has been unambiguously identified. Once sufficient position and/or movement data have been obtained, it is conceivable to indicate, in particular acoustically and/or visually, that only one candidate instrument from the set of candidate instruments remains, whereupon the method for identifying the medical instrument can be discontinued and the geometrical data describing the precalibrated properties of the one remaining instrument can be forwarded to a medical navigation system so as to allow computer-assisted surgery using this instrument.

Another aspect of the present invention relates to a medical instrument identification system comprising a medical tracking system, a computer and a calibration tool as described herein, wherein the calibration tool comprises at least one supporting surface which restricts the relative position and/or the relative movement between the calibration tool and a medical instrument in contact with the at least one supporting surface of the calibration tool.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, in particular electrical, in particular technically generated) signal wave, in particular a digital signal wave, carrying information which represents the program, in particular the aforementioned program, which in particular comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, code or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit or CPU) which executes the computer program elements, and optionally a volatile memory (in particular a random access memory or RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which in particular comprises technical, in particular tangible components, in particular mechanical and/or electronic components. Any device mentioned as such in this document is a technical and in particular tangible device.

It is the function of a tracking marker to be detected by a marker detection device (for example, a camera or an ultrasound receiver or analytical devices such as CT or MRI devices) in such a way that its spatial position (i.e. its spatial location and/or alignment) can be ascertained. The detection device is in particular part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is in particular known to a navigation system and is for example stored in a computer of the navigation system.

The present invention is also directed to a navigation system for computer-assisted surgery. This navigation system preferably comprises the aforementioned computer for processing the data provided in accordance with the data processing method as described in any one of the preceding embodiments. The navigation system preferably comprises a detection device for detecting the position of the detection points which represent the main points and auxiliary points, in order to generate detection signals and to supply the generated detection signals to the computer, such that the computer can determine the absolute main point data and absolute auxiliary point data on the basis of the detection signals received. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

A navigation system, in particular a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) in particular comprises a processor (CPU) and a working memory and advantageously an indicating device for issuing an in signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

In the following, the invention is described with reference to the enclosed figures which represent preferred embodiments of the invention. The scope of the invention is not however limited to the specific features shown in the figures.

FIG. 1 schematically shows a calibration tool such as can be used in connection with the present invention.

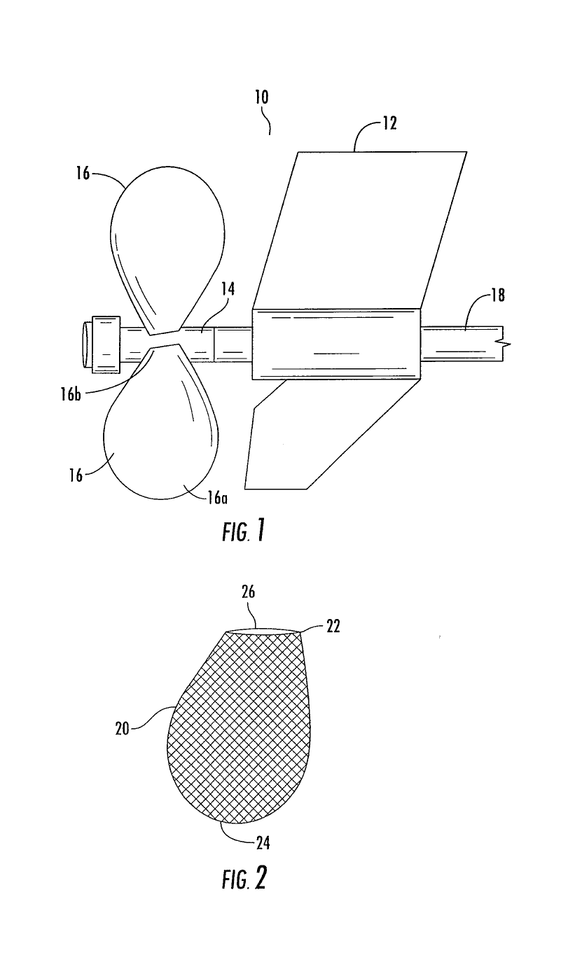

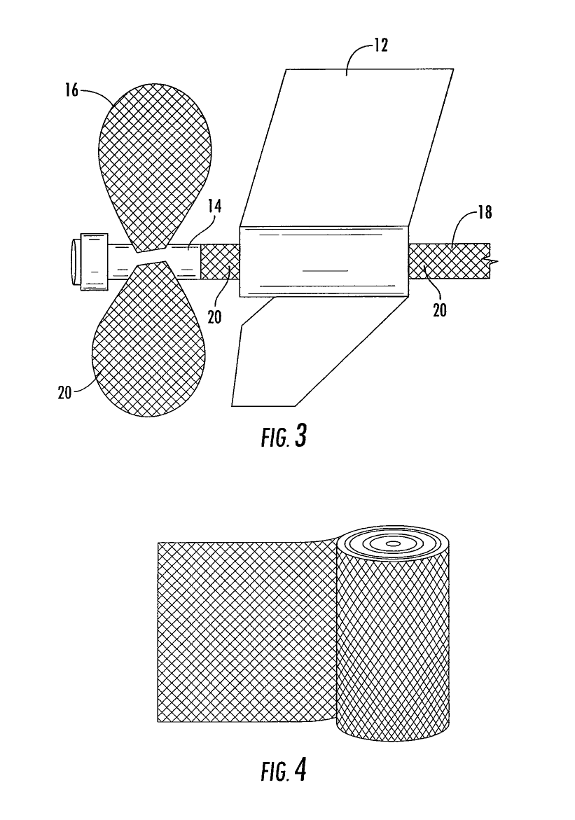

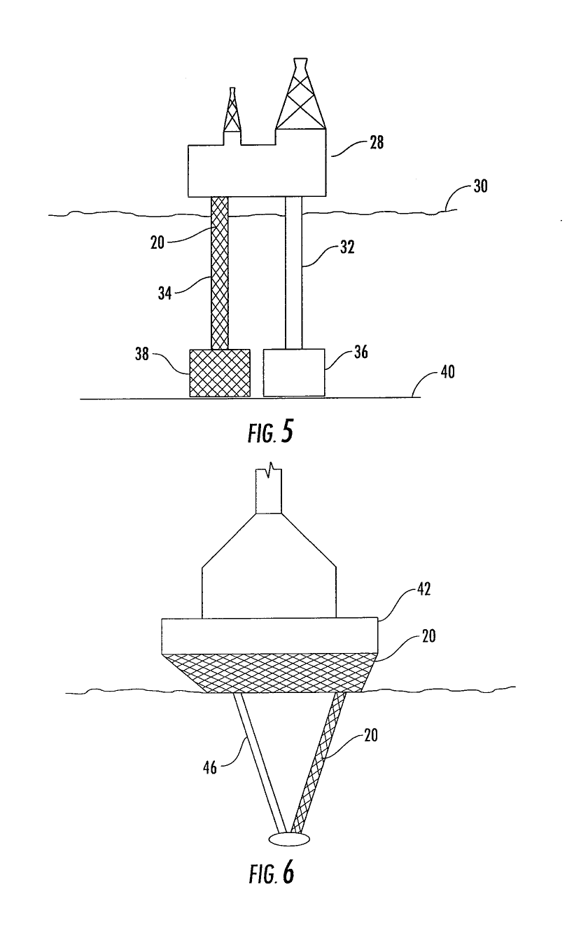

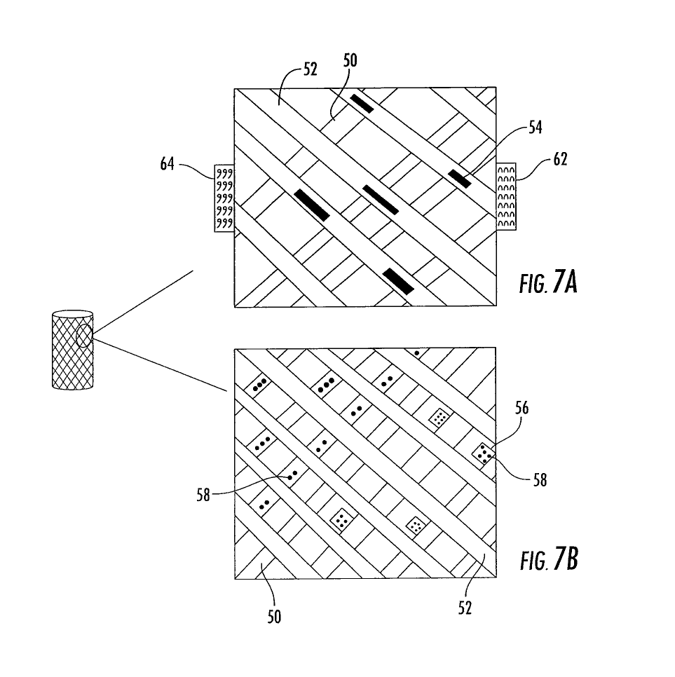

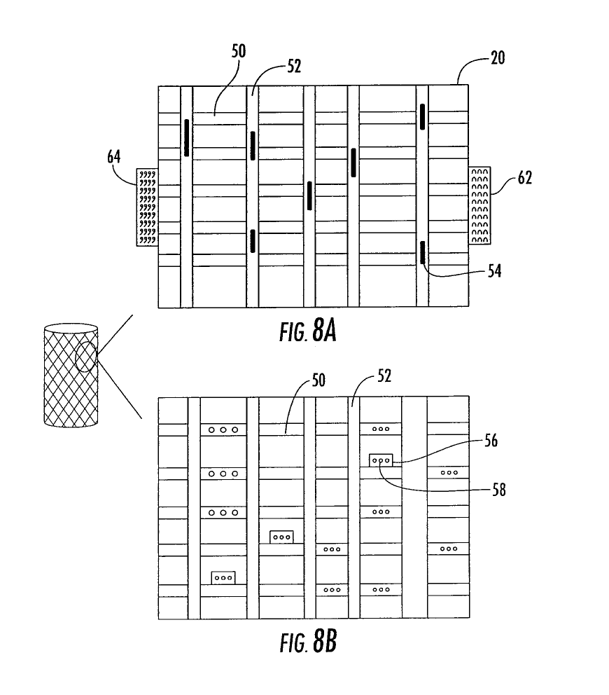

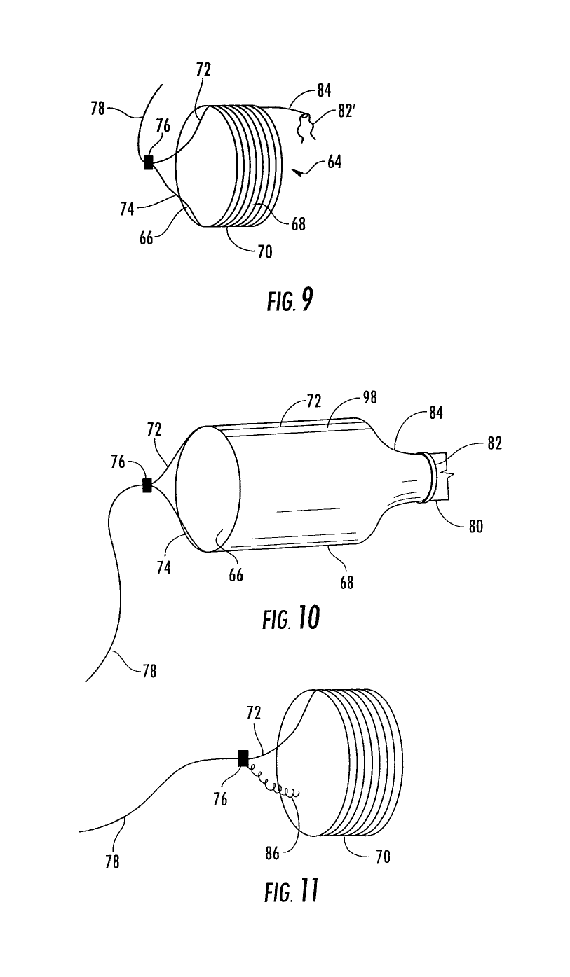

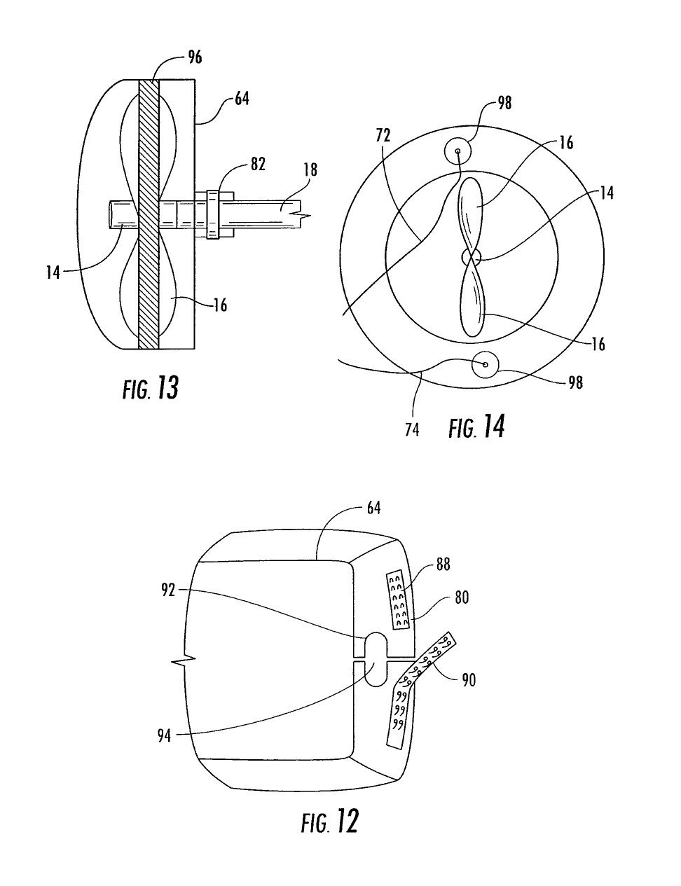

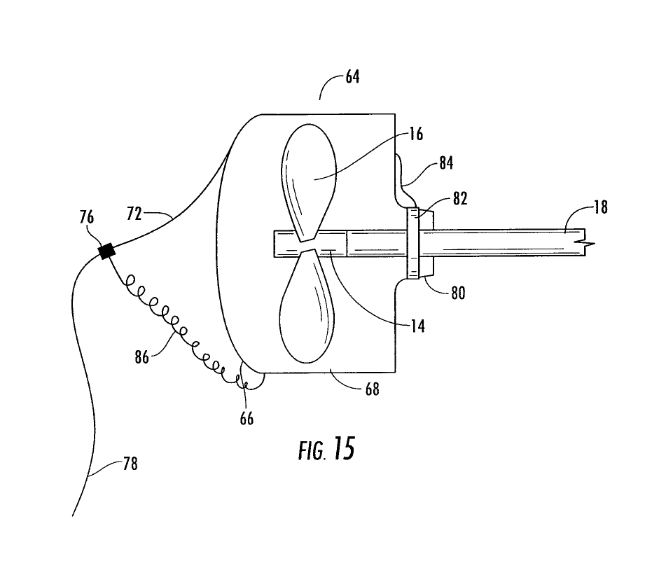

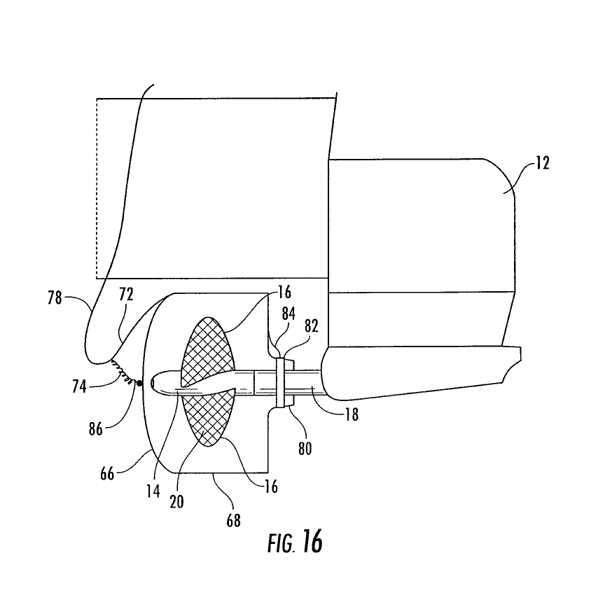

FIG. 2 shows a sequence of relative positions of a medical instrument as it is moved relative to a calibration tool.

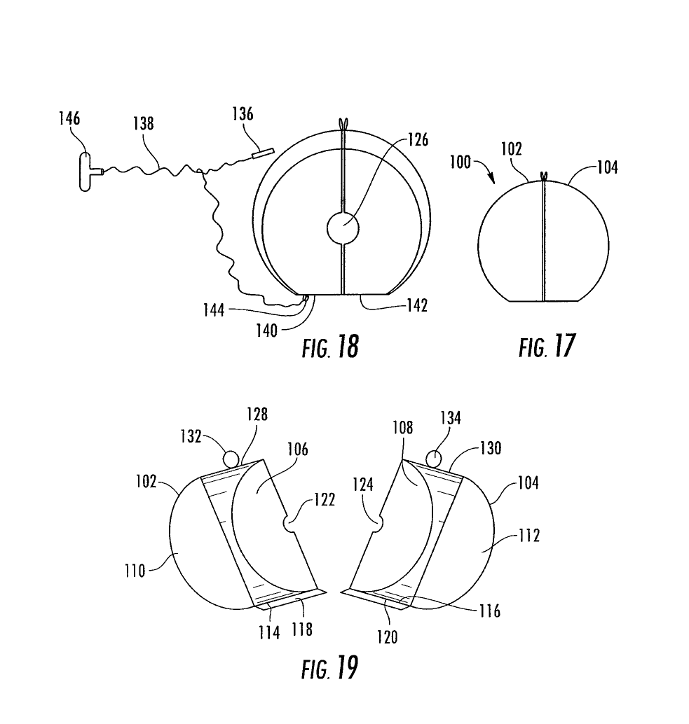

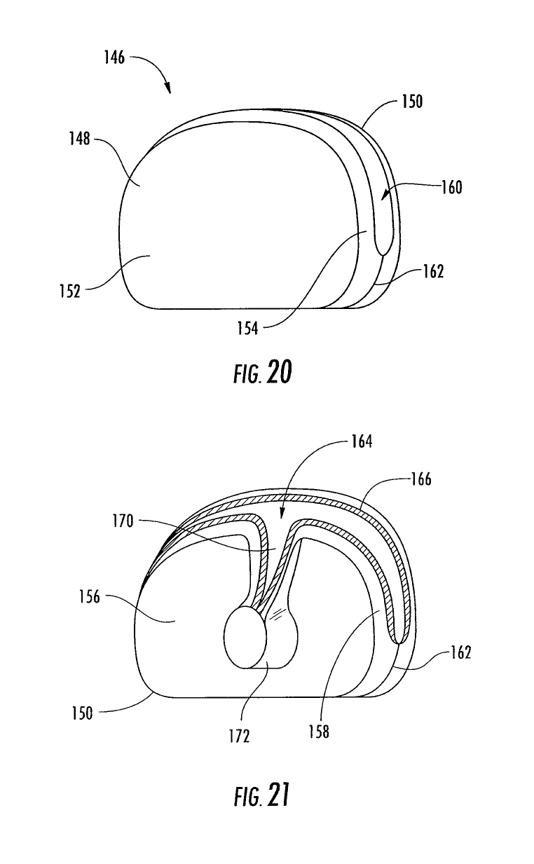

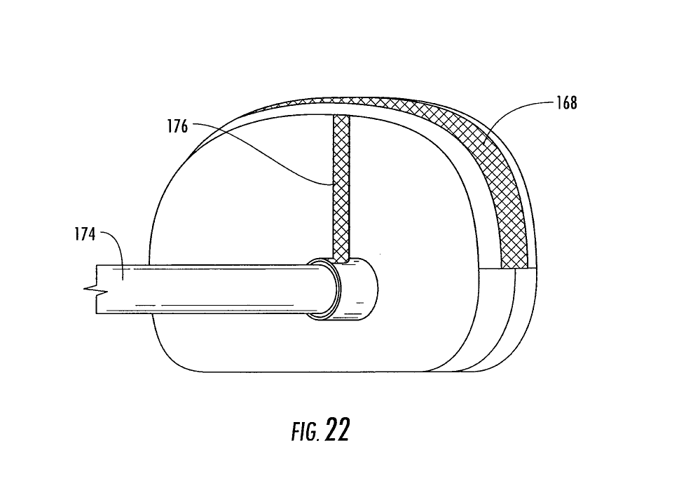

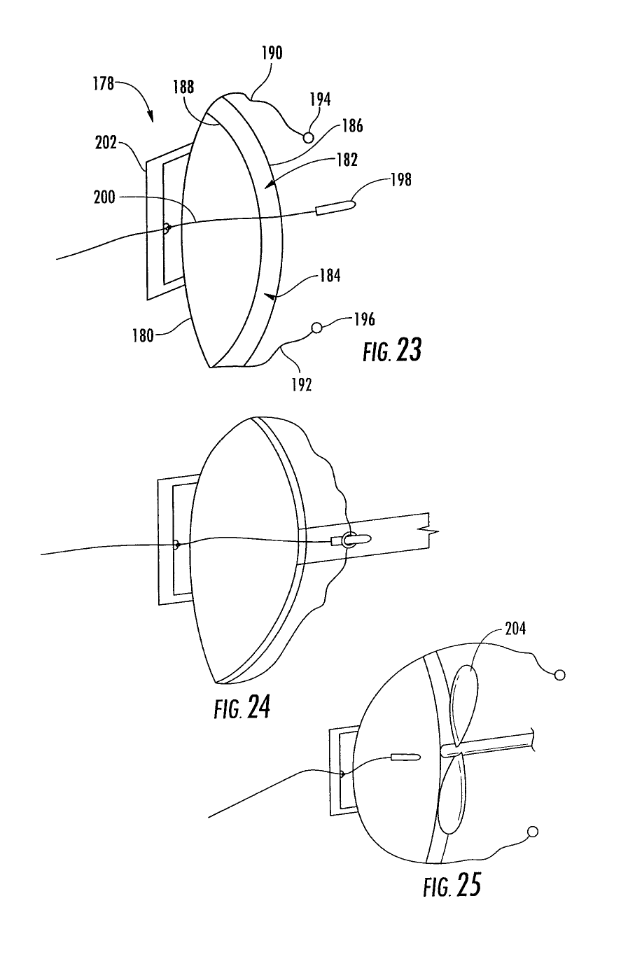

FIG. 3 shows another embodiment of a calibration tool such as can be used in connection with the present invention.

FIG. 1 shows a calibration tool 1, comprising: two angled supporting surfaces 2 which form a V-shaped notch; and an array 3 which comprises three tracking markers and is rigidly attached to the calibration tool 1. The spatial position of each of the three tracking markers is determined by means of a medical tracking system 4 comprising two cameras which are sensitive to infrared light. Once the spatial position of each of the tracking markers has been determined, the spatial position of the calibration tool 1 and consequently also the spatial position of each of the two supporting surfaces 2 is known.

As shown in FIG. 2, an elongate distal section 7 of a medical instrument 5 can be inserted into the notch formed by the supporting surfaces 2 of the calibration tool 1 shown in FIG. 1. Once the distal section 7 has been inserted into the notch, the instrument 5 is moved in a distal direction (indicated by arrows in FIG. 2), during which physical contact between the distal section 7 and each of the supporting surfaces 2 is maintained.

The instrument 5 also has an array 6 comprising three tracking markers (which, for the sake of illustrative clarity, is only shown in the first image of the instrument 5 and not in any of the subsequent images in the sequence shown in FIG. 2) which are fixedly attached to the instrument 5, such that the spatial position of the instrument 5 can be determined by means of the tracking system 4.

Assuming the shape of the distal section 7 is initially unknown, relative position and/or movement data will be obtained as the instrument 5 is moved relative to the calibration tool 1. Initially, only the position of the proximal section of the instrument 5 and the position of the supporting surfaces 2 will be known. As soon as the distal section 7 is inserted into the notch, the spatial position of the part of the distal section 7 which is in contact with the supporting surfaces 2 will also be known. Therefore, each part of the distal section 7 which is moved through the notch, and therefore the relative position of each such part of the distal section 7 relative to the proximal section comprising the tracking marker array 6, will be known to the tracking system 4. The acquired data concerning the shape of the distal section 7 are then compared with the data stored in a database for a plurality of candidate instruments. The greater the geometric data acquired for the instrument 5, the more candidate instruments can be excluded until eventually only one candidate instrument remains, which represents the actual instrument 5 which has been moved through the notch.

It is then possible to transmit data describing the precalibrated instrument, which are necessary for performing computer-assisted surgery, to the navigation system.

The procedure shown in FIG. 2 can be initiated by pressing a start button 10 (see FIG. 1) before the instrument 5 is inserted into the notch of the calibration tool 1, and is completed as soon as there is only one candidate dataset remaining from a plurality of candidate datasets stored in a database on the computer 8.

The movement of the instrument 5 relative to the calibration tool 1 need not of course necessarily be a translational movement, but can instead also be a rotational movement. The distal tip of the distal section 7 could for example be inserted into a tapered recess 9 (shown in an enlarged representation in FIG. 1) and rotated about the point at which the tip is in contact with the recess 9.

As shown in FIG. 3, a calibration tool 1 which is provided can comprise a plurality of supporting surfaces 2 which are configured to abut against the tip of a hollow distal section 7 of a medical instrument 5. Since the calibration tool 1 and the proximal section of the instrument 5 are tracked, geometrical data regarding the diameter of the instrument tip can be obtained by measuring the distance between the tracking marker array 3 on the calibration tool 1 and the tracking marker array 6 on the proximal section of the instrument 5. If the inner diameter of the hollow tip exceeds a predetermined value, the tip will fit over the upper cylindrical section of the calibration tool 1 and abut against the circular supporting surface 2. If the inner diameter of the hollow tip of the instrument 5 does not exceed said predetermined value, the tip will abut against the tapered supporting surface 2 provided within the upper cylindrical section of the calibration tool 1. The lower the value of the outer diameter of the instrument tip, the further down the instrument tip can be inserted onto the tapered supporting surface 2, such that geometrical data regarding the outer diameter of the instrument tip can be obtained by measuring the distance between the tracking reference array 3 and the tracking reference array 6. No relative movement between the instrument 5 and the calibration tool 1 is performed, since the instrument 5 is merely held in a position relative to the calibration tool 1 in which the instrument tip abuts one of the supporting surfaces 2. This enables data regarding both the inner diameter and outer diameter of the hollow tip 7 to be obtained, in order to perform the method of the invention.

1-15. (canceled)

16. A data processing system comprising a computer having a processor configured to execute a computer-implemented medical method for identifying a medical instrument that is tracked by a medical tracking system, the method comprising the steps of:
acquiring, at the processor, relative position and/or movement data which comprise relative position and/or movement information describing a relative position and/or movement between a calibration tool that is tracked by the medical tracking system, which comprises at least one supporting surface, and the medical instrument which is in contact with a side or edge of the at least one supporting surface, respectively;
acquiring, at the processor and from a database comprising datasets containing information about geometry of each of a plurality of candidate instruments, geometry data which comprise geometry information describing at least one geometrical feature for each of the plurality of candidate instruments;
determining, by the processor and on the basis of the relative position and/or movement data and the geometry data, candidate data which comprise candidate information describing at least one candidate instrument from the plurality of candidate instruments, the at least one geometrical feature of which would allow the relative position and/or movement between the medical instrument and the calibration tool.

17. A computer-implemented medical method for identifying a medical instrument that is tracked by a medical tracking system, the method comprising executing, on a processor of a computer, the steps of:
acquiring, at the processor, relative position and/or movement data which comprise relative position and/or movement information describing a relative position and/or movement between a calibration tool that is tracked by the medical tracking system, which comprises at least one supporting surface, and the medical instrument which is in contact with a side or edge of the at least one supporting surface, respectively;
acquiring, at the processor and from a database comprising datasets containing information about geometry of each of a plurality of candidate instruments, geometry data which comprise geometry information describing at least one geometrical feature for each of the plurality of candidate instruments;
determining, by the processor and on the basis of the relative position and/or movement data and the geometry data, candidate data which comprise candidate information describing at least one candidate instrument from the plurality of candidate instruments, the at least one geometrical feature of which would allow the relative position and/or movement between the medical instrument and the calibration tool.

18. The method according to claim 17, wherein the at least one supporting surface restricts the relative position and/or relative movement between the calibration tool and the medical instrument contacting the at least one supporting surface.

19. The method according to claim 17, wherein the medical instrument is moved relative to the calibration tool during the step of acquiring the relative position and/or movement data, and wherein physical contact between the medical instrument and the at least one supporting surface is maintained.

20. The method according to claim 17, wherein the medical instrument is held in a substantially invariant position relative to the calibration tool during the step of acquiring relative position and/or movement data, and wherein physical contact between the medical instrument and the at least one supporting surface is maintained.

21. The method according to claim 17, wherein the step of acquiring the relative position and/or movement data is performed several times.

22. The method according to of claim 21, wherein the step of acquiring the relative position and/or movement data is/are performed until only one candidate instrument from the plurality of candidate instruments remains, the at least one geometrical feature of which would allow all of the relative positions and/or movements performed up until that point.

23. The method according to claim 22, further comprising the step of indicating the at least one candidate instrument for which the at least one geometrical feature which would allow all of the relative positions and/or movements performed up until that point.

24. The method according to claim 22, wherein the method is discontinued as soon as only one candidate instrument from the plurality of candidate instruments remains and/or wherein a signal is outputted which indicates that only one candidate instrument from the plurality of candidate instruments remains, the at least one geometrical feature of which would allow all of the relative positions and/or movements performed up until that point.

25. The method according to claim 22, further comprising the step of identifying the one remaining candidate instrument as the medical instrument on the basis of the candidate data.

26. The method according to claim 17, wherein on the basis of the candidate data, tracking data which comprise tracking information describing features of the medical instrument which would allow the medical instrument to be navigated in a surgical environment are transmitted to a navigation system.

27. The method according to claim 17, wherein the step of acquiring the geometry data involves providing a directory which comprises said geometry data.

28. The method according to claim 17, wherein the at least one relative movement comprises a substantially translational movement.

29. The method according to claim 17, wherein the at least one relative movement comprises a rotational movement.

30. A non-transitory computer-readable program storage medium storing a computer program which, when executed on a processor of a computer or loaded into the memory of a computer, causes the computer to perform a computer-implemented medical method for identifying a medical instrument that is tracked by a medical tracking system, the method comprising the steps of:
acquiring, at the processor, relative position and/or movement data which comprise relative position and/or movement information describing a relative position and/or movement between a calibration tool that is tracked by the medical tracking system, which comprises at least one supporting surface, and the medical instrument which is in contact with a side or edge of the at least one supporting surface, respectively;
acquiring, at the processor and from a database comprising datasets containing information about geometry of each of a plurality of candidate instruments, geometry data which comprise geometry information describing at least one geometrical feature for each of the plurality of candidate instruments;
determining, by the processor and on the basis of the relative position and/or movement data and the geometry data, candidate data which comprise candidate information describing at least one candidate instrument from the plurality of candidate instruments, the at least one geometrical feature of which would allow the relative position and/or movement between the medical instrument and the calibration tool.

31. A computer comprising the non-transitory computer-readable program storage medium according to claim 30.

32. A medical instrument identification system comprising:
the computer according to claim 31;
a medical tracking system; and
a calibration tool that is tracked by the medical tracking system and comprises at least one supporting surface which restricts the relative position and/or the relative movement between the calibration tool and a medical instrument contacting the at least one supporting surface of the calibration tool.
